(19) United States
(12) Patent Application Publication
(10) Pub. No.: US 2008/0269346 A1
(43) Pub. Date: Oct. 30, 2008

(54) B-Blocker Pharmacogenetics in Heart Failure

(75) Inventors: Julie Johnson, Melrose, FL (US); Steven Terra, Ledyard, CT (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Gainesville, FL (US)

(21) Appl. No.: 11/816,480

(22) PCT Filed: Feb. 17, 2006
(86) PCT No.: PCT/US06/05847

§ 371 Date: Aug. 16, 2007

Related U.S. Application Data
(60) Provisional application No. 60/653,815, filed on Feb. 17, 2005.

(51) Int. Cl. A61K 31/137; C12Q 1/68; A61P 9/00

(52) U.S. Cl. 514/651; 435/6

(57) ABSTRACT

Polymorphisms in the beta-1 adrenergic receptor that (1) affect tolerability to beta-blocker treatment or (2) influence responsiveness to beta-blocker treatment are disclosed. Additionally, methods of predicting that an individual would obtain clinical improvement in left ventricular function as a result of β-blocker therapy or identifying at least one individual that would obtain a clinical improvement in left ventricular function as a result of β-blocker therapy are provided. Methods of treating patients characterized by such polymorphisms are also illustrated.

### CROSS-REFERENCE TO RELATED APPLICATION

This application claims the benefit of U.S. Provisional Patent Application 60/653,815, filed Feb. 17, 2005, the disclosure of which is incorporated by reference in its entirety. This application is also a continuation-in-part of U.S. patent application Ser. No. 10/617,846, filed Jul. 10, 2003, pending, which is a continuation of U.S. patent application Ser. No. 10/075,490, filed Feb. 12, 2002, now abandoned, which claims the benefit of U.S. Provisional Patent Applications 60/269,096, filed Feb. 14, 2001, now abandoned, and 60/268,310, filed Feb. 13, 2001, now abandoned. The disclosures of each of these patent applications is hereby incorporated by reference in their entireties.

The subject invention was made with government support under a research project supported by the National Institutes of Health, National Heart, Lung, and Blood Institute (grant #HL68834) and under research support provided to the University of Florida and the University of North Carolina at Chapel Hill by the General Clinical Research Centers program of the Division of Research Resources, National Institutes of Health (grant #RR00082 and grant #RR00046, respectively).

### BACKGROUND OF INVENTION

The syndrome of heart failure is characterized by excessive activation of the sympathetic nervous system leading to release of other neurohormones, structural changes to the left ventricle, and adverse clinical outcomes. [Jessup, M. and Brozena, S. (2003) “Heart failure” N. Engl. J. Med. 348:2007-2018]. The structural changes occurring to the left ventricle include an increase in ventricular dimensions, wall thickness, and left ventricular (LV) mass. [Hall, S. A. et al. (1995) “Time course of improvement in left ventricular function, mass and geometry in patients with congestive heart failure treated with beta-adrenergic blockade” J. Am. Coll. Cardiol. 25:1154-1161; Lowes, B. D. et al. (1999) “Effects of carvedilol on left ventricular mass, chamber geometry, and mitral regurgitation in chronic heart failure” Am. J Cardiol 83:1201-1205]. Collectively, these changes are referred to as remodeling of the ventricle. Importantly, pathologic ventricular remodeling is associated with adverse clinical outcomes in heart failure patients and pharmacological agents that cause reverse remodeling are generally associated with improved clinical outcomes. [Udelson, J. E. and Konstam, M. A. (2002) “Relation between left ventricular remodeling and clinical outcomes in heart failure patients with left ventricular systolic dysfunction” J. Card. Fail. 8:S465-S471].

The β₁-adrenergic receptor (β₁AR) transduces the heightened sympathetic signal in heart failure and contains functional genetic polymorphisms at codons 49 (Ser49Gly) and 389 (Arg389Gly). [Maqbool, A. et al. (1999) “Common polymorphisms of beta1-adrenoceptor: identification and rapid screening assay (letter)” Lancet 353:897]. Some, but not all studies have supported that these polymorphisms have functional consequences. One ex vivo study using atrial tissue from patients undergoing surgery demonstrated that tissue from Arg389 homozygous patients had greater inotropic potency of norepinephrine and greater norepinephrine-stimulated cAMP accumulation. [Sandilands, A. J. et al. (2003) “Greater inotropic and cyclic AMP responses evoked by noradrenaline through Arg389 β₁-adrenoceptors versus Gly389 β₁-adrenoceptors in isolated human atrial myocardium” Br. J. Pharmacol. 138:386-392]. A similar study of right atrial tissue showed no differences in norepinephrine mediated effects by codon 49 or 389 genotype. [Molenaar, P, et al. Conservation of the cardiostimulant effects of (−)-norepinephrine across Ser49Gly and Gly389Arg Beta1-adrenergic receptor polymorphisms in human right atrium in vitro]. In vitro mutagenesis studies revealed the Arg389 form of the β₁AR has greater basal and agonist-mediated adenylyl cyclase activity than the Gly389 form. [Mason, D. A. et al. (1999) “A gain-of-function polymorphism in a G-protein coupling domain of the human β₁-adrenergic receptor” J. Biol. Chem. 274:12670-12674]. The Arg389 allele also down-regulates to a significantly lower extent than Gly389, and transgenic mice expressing Arg389 develop greater impairment of LV function over time compared with Gly389 mice. [Mialet, P. J. et al. (2003) “Beta 1-adrenergic receptor polymorphisms confer differential function and predisposition to heart failure” Nat. Med. 9:1300-1305]. At codon 49, the Ser49 variant is resistant to agonist mediated down regulation and transfected cells with the Ser49 variant produce markedly greater cAMP concentrations compared to Gly49. [Rathz, D. A. et al. (2002) “Amino acid polymorphism of the human beta 1-adrenergic receptor affect agonist-promoted trafficking” J. Cardiovasc. Pharmacol. 39:155-160.].

When first put on the market, β-adrenergic blocking agents were considered to be contraindicated in patients with heart failure. [Lassig, et al. (2001) “Beta-blockers and heart failure” J. Clin. Basic Cardiol. 4(1):11-14]. A failing heart was originally thought to profit from sympathetic stimulation, but it is now recognized that chronic activation of the sympathetic nervous system damages the myocardium. Consistent with this new understanding, the β-blockers metoprolol succinate, bisoprolol, and carvedilol have been shown to significantly reduce morbidity and mortality in patients with heart failure. [MERIT-HF Study Group (1999) “Effects of metoprolol CR/XL in chronic heart failure: Metoprolol CR/XL randomised intervention trial in congestive heart failure (MERIT-HF)” Lancet 353:2001-2007; CIBIS-II Investigators and Committees (1999) “The cardiac insufficiency bisoprolol study II (CIBIS-II): A randomised trial” Lancet 353:9-13; Packer, M. et al. (2001) “Carvedilol prospective randomized cumulative survival study group. Effect of carvedilol on survival in severe chronic heart failure” N. Engl. J. Med. 344:1651-1658]. Accordingly, β-blockers are now recommended in consensus guidelines as standard therapy for management of patients with systolic heart failure. [Hunt, S. A. et al. (2001) “ACC/AHA guidelines for the evaluation and management of chronic heart failure in the adult: executive summary. A report of the American College of Cardiology/American Heart Association Task Force on Practice Guidelines (Committee to revise the 1995 Guidelines for the Evaluation and Management of Heart Failure)” J. Am. Coll. Cardiol. 38:2101-2113].

It has been demonstrated that β-blocker therapy can partially reverse LV hypertrophy and abnormal geometry [Hall, S. A. (1995) “Time course of improvement in left ventricular function, mass and geometry in patients with congestive heart failure treated with beta-adrenergic blockade” J. Am. Coll. Cardiol. 25:1154-1161; Lowes, B. D. et al. (1999) “Effects of carvedilol on left ventricular mass, chamber geometry, and mitral regurgitation in chronic heart failure” Am. J Cardiol 83:1201-1205; Doughty, R. N. et al. (1997) “Left ventricular remodeling with carvedilol in patients with congestive heart failure due to ischemic heart disease. Australia-New Zealand Heart Failure Research Collaborative Group” J. Am. Coll. Cardiol. 29:1060-1066], and these effects could be related to the reductions in clinical endpoints. A meta-analysis found that drugs that produced reductions in ventricular volumes, such as ACE inhibitors and β-blockers produced reductions in mortality. [Konstam, M. A. et al. (2003) “Ventricular remodeling in heart failure: a credible surrogate endpoint” J. Card Fail. 9:350-353]. Conversely, drugs causing a null or increase in ventricular volumes either had no impact on mortality or increased mortality, respectively. [Konstam, M. A. et al. (2003) “Ventricular remodeling in heart failure: a credible surrogate endpoint” J. Card. Fail. 9:350-353]. Patients with the largest increase in ejection fraction (EF) following β-blocker therapy appear to have the best prognosis. [Metra, M. et al. (2003) “Marked improvement in left ventricular ejection fraction during long-term β-blockade in patients with chronic heart failure: clinical correlates and prognostic significance” Am. Heart J. 145:292-299]. In the CIBIS-II study with bisoprolol, increased LVESD was a significant predictor of death and hospitalization for worsening heart failure. [Lechat, P. et al (2001) “Heart rate and cardiac rhythm relationships with bisoprolol benefit in chronic heart failure in CIBIS II trial” Circulation 103:1428-1433]. Left ventricular end-diastolic diameter has also been shown to be an independent predictor of sudden cardiac death. [La Rovere, M. T. et al. (2003) “Short-term heart rate variability strongly predicts sudden cardiac death in chronic heart failure patients” Circulation 107:565-570]. Furthermore, another meta-analysis found that carvedilol increased the EF of heart failure patients significantly more than metoprolol, [Packer, M. et al. (2001) “Comparative effects of carvedilol and metoprolol on left ventricular ejection fraction in heart failure: results of a meta-analysis” Am. Heart J. 141:899-907] an observation consistent with the findings in the COMET study in which carvedilol-treated patients had a lower risk of death compared to immediate-release metoprolol. [Poole-Wilson, P. A. et al. (2003) “Comparison of carvedilol and metoprolol on clinical outcomes in patients with chronic heart failure in the Carvedilol or Metoprolol European Trial (COMET): Randomised controlled trial” Lancet 362:7-13]. Thus, while there are undoubtedly a myriad of mechanisms responsible for the mortality benefits from β-blockers, it seems likely that an improvement in LV function and remodeling accounts for much of the clinical benefit.

Despite the overwhelming benefits observed in clinical trials, β-blocker utilization has been less than optimal. A recent European survey demonstrated that only 34% of heart failure patients were receiving β-blockers, and among patients with left ventricular dysfunction, β-blockers were used in only 20%. [Cleland, J. G. F. et al. (2002) “Management of heart failure in primary care (the IMPROVEMENT of Heart Failure Programme): An international survey” Lancet 360:1631-16391. Among the reasons for the relatively low use of β-blockers is the initial risk of cardiac decompensation or worsening heart failure that can occur upon initiation of therapy in certain patients, particularly those with more advanced heart failure. [Gottlieb, S. S. et al. (2002) “Tolerability of beta-blocker initiation and titration in the Metoprolol CR/XL Randomised Intervention Trial in Congestive Heart Failure (MERIT-HF)” Circulation 105:1182-1188]. The initiation of β-blocker therapy also requires frequent clinic visits for dose titration and diligent management of fluid balance and concomitant medications to decrease the risk of cardiac deterioration. Thus, identification of factors that increase susceptibility to initial decompensation might enhance the uptake of this therapy in clinical practice.

β-blockers presumably produce their beneficial effects by blunting the detrimental effects of sympathetic nervous system activation. Through this mechanism, β-blockers lead to improvements in ejection fraction (EF) and other indices of LV function along with reductions in clinical endpoints. However, large variability exists in the improvements in LV function and responsiveness to β-blocker therapy varies widely amongst individuals (for example, a study comparing carvedilol and metoprolol revealed 95% confidence intervals for the change in LVEF of −11.1 to +32.9 for carvedilol and −8.2 to +22.6 for metoprolol. [Metra, M. et al. (2000) “Differential effects of β-blockers in patients with heart failure” Circulation 102:546-551]). Genetic polymorphism may be one cause of such variability. For example, the genetic polymorphisms in the β₁AR have been shown to have a role in the anti-hypertensive response to β-blockers. [Johnson, J. A. et al. (2003) “Beta 1-adrenergic receptor polymorphisms and antihypertensive response to metoprolol” Clin. Pharmacol. Ther. 74:44-52]. Few studies have investigated the influence of the codon 49 and 389 polymorphisms together. In the one study where this was done (Johnson, above), the most responsive individuals contained two copies of Ser49Arg389.

In African Americans, the Arg389Gly polymorphism and the common insertion/deletion (Ins/Del) polymorphism in the α_{2C}-adrenergic receptor gene (ADRA2C) have been associated to act synergistically to increase the risk for heart failure [Small et al. (2002) “Synergistic polymorphisms of beta1- and alpha2C-adrenergic receptors and the risk of congestive heart failure” N Engl J. Med., 347:1135-42]. With respect to the common insertion/deletion polymorphism, a 12-nucleotide deletion (Del) results in the deletion of four amino acids (322-325, Gly-Ala-Gly-Pro) in the third intracellular loop of the receptor and a substantial loss of agonist-mediated receptor function [Small et al. (2000) “A four amino acid deletion polymorphism in the third intracellular loop of the human alpha 2C-adrenergic receptor confers impaired coupling to multiple effectors” J Biol Chem., 275:23059-64; Feng et al. (2001) “An in-frame deletion in the alpha(2C) adrenergic receptor is common in African-Americans” Mol Psychiatry, 6:168-72].

### BRIEF SUMMARY OF THE INVENTION

In one aspect the present invention provides a method for predicting the tolerability of a subject to beta-blocker treatment, where said method comprises detection of a polymorphism in a nucleic acid encoding an element of at least one β-adrenergic receptor from the subject. The presence of the polymorphism is correlated with a need for an increase in other heart failure medications prescribed during beta-blocker titration thereby identifying the subject as especially likely to require additional intervention concomitant with initiation of beta-blocker therapy. In various embodiments, the polymorphisms detected include those at codons 49 (Ser49Gly) and 389 (Arg389Gly) of the gene encoding ADRB1 (β₁AR).

In another aspect the present invention provides a method for predicting the responsiveness of a subject to beta-blocker treatment, where said method comprises detection of a polymorphism in a nucleic acid encoding an element of at least one β-adrenergic receptor from the subject. The presence of the polymorphism is correlated with the degree of reverse remodeling associated with beta-blocker treatment thereby predicting the responsiveness of the subject to beta-blocker therapy. In various embodiments, the polymorphisms detected include those at codons 49 (Ser49Gly) and 389 (Arg389Gly) of the gene encoding ADRB1 (β₁AR).

In yet another aspect the present invention also provides a method for identifying an allele correlated with tolerability or responsiveness to beta-blocker treatment, comprising detection of the presence of a polymorphism in a nucleic acid encoding at least one component of a β-adrenergic receptor from a subject, wherein the presence of the polymorphism is correlated with tolerability or responsiveness to beta-blocker treatment. This identifies the allele as being correlated with tolerability or responsiveness to beta-blocker treatment. In various embodiments, the polymorphisms detected include those at codons 49 (Ser49Gly) and 389 (Arg389Gly) of the gene encoding ADRB1 (β₁AR).

A method of selecting at least one individual for treatment with β-blockers for the improvement of left ventricular ejection fraction response is also provided by the subject application. This method comprises genotyping the β₁ adrenergic receptor (β₁AR) and the α_{2C} adrenergic receptor (ADRA2C) gene of at least one individual. Following the genotyping step, one determines whether the genotype of the β₁AR gene of said at least one individual is Arg389Arg and the genotype of the ADRA2C gene is DEL (e.g., whether the ADRA2C receptor gene contains a 12-nucleotide deletion (DEL) that results in the deletion of four amino acids (322-325, Gly-Ala-Gly-Pro) in the third intracellular loop of the receptor). Finally, the method selects said at least one individual for treatment with β-blockers if the individual exhibits the ARG389ARG and DEL genotype.

Also provided by the subject application is a method of predicting that an individual would obtain clinical improvement in left ventricular function as a result of β-blocker therapy or identifying at least one individual that would obtain a clinical improvement in left ventricular function as a result of β-blocker therapy. This method comprises genotyping the β₁ adrenergic receptor (β₁AR) gene and the α_{2C} adrenergic receptor (ADRA2C) gene of at least one individual. Following the genotyping step, one determines whether the genotype of the β₁AR gene of said at least one individual is Arg389Arg and the genotype of the ADRA2C gene is DEL. One then identifies those individuals that exhibit the ARG389ARG and DEL genotype as being expected to obtain a clinical improvement in left ventricular function as a result of β-blocker therapy.

The subject application also provides a method of treating at least one individual having impaired left ventricular function comprising: a) genotyping the β₁ adrenergic receptor (β₁AR) gene and the α_{2C} adrenergic receptor (ADRA2C) gene of at least one individual; b) determining whether the genotype of the β₁AR gene of said at least one individual is Arg389Arg and the genotype of the ADRA2C gene is DEL; and c) administering a therapeutically effective amount of a β-blocker medication to said individual if the genotype of the β₁AR gene of said at least one individual is Arg389Arg and the genotype of the ADRA2C gene is DEL. Non-limiting examples of beta blocker medications that can be used in this aspect of the invention include, and are not limited to, atenolol, betaxolol, bisoprolol, metoprolol, long-acting metoprolol, carvedilol, carteolol, nadolol, penbutolol, propranolol, long-acting propranolol, timolol, labetalol, salts thereof, and combinations thereof. In certain embodiments, metoprolol, carvedilol, bisoprolol or combinations thereof can be. β-blocker medications are administered in amounts sufficient to improve left ventricular function in the individual.

### BRIEF DESCRIPTION OF THE TABLES

Table 1 depicts baseline demographic characteristics as stratified by β₁AR genotype for patients studied for metoprolol tolerability.

Table 2 illustrates the risk of decompensated heart failure as differentiated by β₁AR genotype for patients studied for metoprolol tolerability.

Table 3 shows the impact of β₁-AR genotype on the 6-minute walk distance, quality-of-life score, hemodynamics, and dose of metoprolol CR/XL at the end of the titration period for patients studied for metoprolol tolerability.

Table 4 presents baseline characteristics according to codon 389 genotype for patients studied for ventricular remodeling.

Table 5 portrays heart rate, blood pressure and left ventricular changes stratified by codon 389 genotype for patients studied for ventricular remodeling.

Table 6 provides the multivariate analysis of factors influencing the change in ejection fraction for patients studied for ventricular remodeling.

Table 7 describes the multivariate analysis of factors influencing the change in LV end-diastolic diameter for patients studied for ventricular remodeling.

Table 8 provides baseline characteristics and genotyping results for individuals studied with respect to improvement in left ventricular function.

Table 9 provides multiple regression analysis of factors influencing the treatment ejection fraction.

Table 10 illustrates changes in EF stratified by genotype combination of α_{2C}-adrenergic receptor Ins/Del polymorphism and β₁-adrenergic receptor Arg389Gly polymorphism.

### BRIEF DESCRIPTION OF THE FIGURES

FIG. 1 illustrates the percentage of patients requiring increases in concomitant heart failure medications during metoprolol CR/XL titration according to β₁AR diplotype. CR/XL Titration According to DIM Diplotype. SR/SG=Ser49Ser+Arg389Gly; SR/SR=Ser49Ser+Arg389Arg; SR/GR=Ser49Gly+Arg389Arg; SG/OR=Ser49Gly+Arg389Gly. P=0.01 between groups.

FIG. 2 depicts the effect of β₁AR codon 389 polymorphism on LV remodeling during treatment with metoprolol CR/XL. Key: LVEDD=Left ventricular end-diastolic diameter; LVESD=Left ventricular end-systolic diameter.

FIG. 3 shows the effect of β₁AR diplotype on changes in LV end-diastolic diameter during treatment with metoprolol CR/XL. Data are Least Squares Means. Key: SR/SG=Ser49Ser+Arg389Gly; SR/SR=Ser49Ser+Arg389Arg; SR/GR=Ser49Gly+Arg389Arg; SG/GR=Ser49Gly+Arg389Gly.

FIG. 4 shows the effect of α_{2C}-adrenergic receptor Ins/Del polymorphism on changes in heart rate in heart failure patients.

FIG. 5 shows the effect of the combination of α_{2C}-adrenergic receptor Ins/Del polymorphism and β₁-adrenergic receptor Arg389Gly polymorphism on ejection fraction.

### DETAILED DISCLOSURE

In one aspect the present invention provides a method for predicting the tolerability of a subject to beta-blocker treatment, where said method comprises detection of a polymorphism in a nucleic acid encoding an element of at least one β-adrenergic receptor from the subject. The presence of the polymorphism is correlated with an increase in other heart failure medications prescribed during beta-blocker titration thereby identifying the subject as especially likely to require additional intervention concomitant with initiation of beta-blocker therapy. In various embodiments, the polymorphisms detected include those at codons 49 (Ser49Gly) and 389 (Arg389Gly) of the gene encoding ADRB1 (β₁AR).

In another aspect the present invention provides a method for predicting the responsiveness of a subject to beta-blocker treatment, where said method comprises detection of a polymorphism in a nucleic acid encoding an element of at least one β-adrenergic receptor from the subject. The presence of the polymorphism is correlated with the degree of reverse remodeling associated with beta-blocker treatment thereby predicting the responsiveness of the subject to beta-blocker therapy. In various embodiments, the polymorphisms detected include those at codons 49 (Ser49Gly) and 389 (Arg389Gly) of the gene encoding ADRB1 (β₁AR).

In yet another aspect the present invention also provides a method for identifying an allele correlated with tolerability or responsiveness to beta-blocker treatment, comprising detection of the presence of a polymorphism in a nucleic acid encoding at least one component of a β-adrenergic receptor from a subject, wherein the presence of the polymorphism is correlated with tolerability or responsiveness to beta-blocker treatment. This identifies the allele as being correlated with tolerability or responsiveness to beta-blocker treatment. In various embodiments, the polymorphisms detected include those at codons 49 (Ser49Gly) and 389 (Arg389Gly) of the gene encoding ADRB1 (β₁AR).

The presence of a polymorphism is identified by determining the nucleic acid sequence of at least a region of a gene encoding a β-adrenergic receptor, or receptor associated protein, according to standard molecular biology protocols well known in the art, for example, as described in Sambrook et al. [Sambrook, J. et al. (1989). “Molecular Cloning: A Laboratory Manual, 2^{nd} ed.”, Cold Spring Harbor Laboratory Press, Cold Spring Harbor, N.Y., pp. 9-16] or as set forth in the Examples provided herein. One skilled in the art will appreciate that the gene or a region thereof can contain one or more polymorphisms associated with tolerability or responsiveness to beta-blocker treatment. Examples of methods of nucleic acid detection known in the art, such as nucleic acid sequencing, polymerase chain reaction (PCR) with or without restriction fragment length polymorphism (RFLP) analysis, Southern and Northern blot analysis, ligase chain reaction, and PCR reaction of specific alleles (PASA) can be utilized to enhance the subject assay and are described for example in Sambrook et al. [Sambrook, J. et al. (1989) “Molecular Cloning: A Laboratory Manual, 2^{nd} ed.”, Cold Spring Harbor Laboratory Press, Cold Spring Harbor, N.Y., pp. 9-16].

Other techniques such as isothermal, single-cycle amplification technique, termed the self-sustained sequence replication (3SR) system, can discriminate between a wild-type or mutant sequence at any particular residue. Because this amplification method generates a predominance of one strand of single-stranded RNA, direct sequencing is possible.

One method for detecting a nucleic acid encoding at least one β-adrenergic receptor variant or a variant receptor associated protein can comprise contacting the pre-selected portion of a whole blood sample with at least one detectable nucleic acid probe that is selective for the nucleic acid encoding at least one β-adrenergic receptor variant or a variant receptor associated protein under conditions favorable for promoting hybridization of the probe. The method then detects the presence of the hybridization between the probe and the nucleic acid, thereby detecting the presence of the nucleic acid encoding at least one β-adrenergic receptor variant or a variant receptor associated protein. Conditions favorable for promoting hybridization of a particular probe to a nucleic acid can vary depending upon the sequence of the nucleic acid being detected or the type of probe utilized. However, such conditions are generally known in the art and will be apparent to the skilled artisan. Thus, one can merely adapt the procedures set forth in the art to suit the present methods.

In some embodiments, the invention further provides for the treatment of individuals identified as being either (1) at greater or lesser risk for complications associated with the initiation of beta-blocker therapy or (2) more likely or less likely to experience beneficial effects from beta-blocker therapy. In this aspect of the invention, individuals identified as having a greater risk for complications associated with the initiation of beta-blocker therapy can be monitored more closely during any such initiation, while individuals identified as having a lesser risk can be monitored less closely during any such initiation. Individuals identified as likely to be less responsive to beta-blocker therapy can be treated preferentially or in greater proportion with alternative art-recognized non-beta-blocker therapies, while individuals identified as likely to be more responsive to beta-blocker therapy can be treated preferentially or in greater proportion with beta-blocker therapy. Non-limiting examples of beta blocker medications typically used in the practice of this invention can be selected from the group consisting of atenolol, betaxolol, bisoprolol, metoprolol, long-acting metoprolol, carvedilol, carteolol, nadolol, penbutolol, propranolol, long-acting propranolol, timolol, labetalol, salts thereof, and combinations thereof by the skilled practitioner. In certain embodiments, metoprolol, carvedilol, bisoprolol or combinations thereof are used for the treatment of a patient suffering from heart failure.

Additional or alternative medications (“heart failure medications”) for use in combination therapy of heart failure patients include, and are not limited to, diuretics, angiotensin converting enzyme (ACE) inhibitors, angiotensin receptor blockers salts thereof, and combinations of the aforementioned medications can be used for treatment by the skilled practitioner. Diuretics include, and are not limited to, torsemide, bendroflumethiazide, benzthiazide, chlorothiazide, cyclothiazide, hydrochlorothiazide, hydroflumethiazide, indapamide, methylclothiazide, metolazone, polythiazide, quinethazone, trichlormethiazide, bumetanide, ethacrynic acid, furosemide, amiloride, spironolactone, triamterene. ACE inhibitors include, and are not limited to, captopril, enalapril, fosinopril, lisinopril, ramipril, trandolapril and salts thereof. Angiotensin receptor blockers include, are not limited to losartan, irbesartan, candesartan, valsartan, and salts thereof.

The subject invention also provides methods of reducing delays in determining the most appropriate therapy or combination of therapies for an individual, wherein said methods comprise:
a) genotyping
   1) the β₁ adrenergic receptor (β₁AR) of said individual at codon 49, wherein the presence of the Gly49 carrier genotype is indicative of (i) decreased risk for complications associated with the initiation of beta-blocker therapy or (ii) increased responsiveness to beta-blocker therapy; or
   2) the β1 adrenergic receptor (β₁AR) of said individual at codon 49, wherein the presence of the Ser49 homozygous genotype is indicative of (i) increased risk for complications associated with the initiation of beta-blocker therapy or (ii) decreased responsiveness to beta-blocker therapy; or
   3) the β₁ adrenergic receptor (β₁AR) of said individual at codon 389, wherein the presence of the Gly389 carrier genotype is indicative of (i) increased risk for complications associated with the initiation of beta-blocker therapy or (ii) decreased responsiveness to beta-blocker therapy; or
   4) the β₁ adrenergic receptor (β₁AR) of said individual at codon 389, wherein the presence of the Arg389 homozygous genotype is indicative of (i) decreased risk for complications associated with the initiation of beta-blocker therapy or (ii) increased responsiveness to beta-blocker therapy; and
b) providing, on the basis of the observed genotype, an appropriate therapeutic agent, wherein beta blocker medications are prescribed preferentially or in greater proportion to an individual having a Gly49 carrier genotype or an Arg 389 homozygous genotype and wherein patients having a Ser49 homozygous genotype or Gly389 carrier genotype are preferentially or in greater proportion prescribed alternative non-beta blocker therapeutics.

Any art-known methods of indirectly inferring genotype are herein regarded as equivalent to genotyping. For example, sequencing or otherwise determining the composition of messenger RNA or expressed protein is herein regarded as equivalent to genotyping.

A method of selecting at least one individual for treatment with β-blockers for the improvement of left ventricular ejection fraction response is also provided by the subject application. This method comprises genotyping the β₁ adrenergic receptor (β₁AR) and the α_{2C} adrenergic receptor (ADRA2C) gene of at least one individual. Following the genotyping step, one determines whether the genotype of the β₁AR gene of said at least one individual is Arg389Arg and the genotype of the ADRA2C gene is DEL (e.g., whether the ADRA2C receptor gene contains a 12-nucleotide deletion (DEL) that results in the deletion of four amino acids (322-325, Gly-Ala-Gly-Pro) in the third intracellular loop of the receptor). Finally, the method selects said at least one individual for treatment with β-blockers if the individual exhibits the ARG389ARG and DEL genotype.

Also provided by the subject application is a method of predicting that an individual would obtain clinical improvement in left ventricular function as a result of β-blocker therapy or identifying at least one individual that would obtain a clinical improvement in left ventricular function as a result of β-blocker therapy. This method comprises genotyping the β₁ adrenergic receptor (β₁AR) gene and the α_{2C} adrenergic receptor (ADRA2C) gene of at least one individual. Following the genotyping step, one determines whether the genotype of the β₁AR gene of said at least one individual is Arg389Arg and the genotype of the ADRA2C gene is DEL. One then identifies those individuals that exhibit the ARG389ARG and DEL genotype as being expected to obtain a clinical improvement in left ventricular function as a result of β-blocker therapy.

The subject application also provides a method of treating at least one individual having impaired left ventricular function comprising: a) genotyping the β₁ adrenergic receptor (β₁AR) gene and the α_{2C} adrenergic receptor (ADRA2C) gene of at least one individual; b) determining whether the genotype of the β₁AR gene of said at least one individual is Arg389Arg and the genotype of the ADRA2C gene is DEL; and c) administering a therapeutically effective amount of a β-blocker medication to said individual if the genotype of the β₁AR gene of said at least one individual is Arg389Arg and the genotype of the ADRA2C gene is DEL. Non-limiting examples of beta blocker medications that can be used in this aspect of the invention include, and are not limited to, atenolol, betaxolol, bisoprolol, metoprolol, long-acting metoprolol, carvedilol, carteolol, nadolol, penbutolol, propranolol, long-acting propranolol, timolol, labetalol, salts thereof, and combinations thereof. In certain embodiments, metoprolol, carvedilol, bisoprolol or combinations thereof can be. β-blocker medications are administered in amounts sufficient to improve left ventricular function in the individual.

It should be understood that the embodiments and Examples described herein are for illustrative purposes only and that various modifications or changes in light thereof will be suggested to persons skilled in the art and are to be included within the spirit and purview of this application. All patents, patent applications, provisional applications, and publications referred to or cited herein are incorporated by reference in their entirety to the extent they are not inconsistent with the explicit teachings of this specification.

### EXAMPLE 1

### ADRB1 (β₁AR Gene) and Tolerability to Metoprolol CR/XL in Heart Failure

Patients were recruited for this prospective study from the University of Florida and University of North Carolina Heart Failure Clinics. All clinicians involved in clinical care of these patients were blinded to the genotypic data and all investigators determining the genotypes were blinded to the patients' clinical course. The study was approved by the Institutional Review Board at each institution and each patient gave written informed consent for participation in the study. The study population included patients aged ≧18 years with symptoms of heart failure due to an ischemic or non-ischemic etiology in NYHA functional class II-III, and with an ejection fraction ≦40%. Patients were not eligible for enrollment if they were receiving β-blocker therapy. Other exclusion criteria were NYHA functional class IV, systolic blood pressure <90 mm Hg, heart rate <55 beats/minute, bronchospastic lung disease, >1^{st} degree heart block, active myocarditis, hypertrophic obstructive cardiomyopathy, and treatment with sotalol.

After obtaining informed consent, a 5-mL blood sample for isolation of genomic DNA was collected. Baseline evaluations included assessment of sitting vital signs and body weight, a 6-minute walk test to assess submaximal exercise tolerance, [Guyatt, G. H. et al. (1985) “The 6-minute walk: A new measure of exercise capacity in patients with chronic heart failure” Can. Med. Assoc. J 132:919-923] and the Minnesota living with heart failure (MLWHF) questionnaire [Rector, T. S. and Cohn, J. N. (1992) “Assessment of patient outcome with the Minnesota living with heart failure questionnaire: Reliability and validity during a randomized, double-blind, placebo-controlled trial of pimobendan” Am. Heart J. 124:1017-1025]. After completion of baseline studies, patients began metoprolol CR/XL at a dose of 12.5 (NYHA class III) or 25 mg/day (NYHA class II) depending on functional class. The dose was then doubled on a biweekly basis up to 200 mg/day or maximum tolerated dose. Thus, the time for up-titration period was 8-10 weeks depending on the starting dose of metoprolol CR/XL. The dosing regimen of metoprolol CR/XL could be adjusted by the clinician. Concomitant medication changes were allowed during the titration period but were discouraged except in response to a change in symptoms. The 6-minute walk test, functional class assessment, MLWHF questionnaire, vital sign measurements and body weight were repeated after two weeks on each dose of metoprolol CR/XL.

Genomic DNA was isolated from whole blood using a commercially available kit (Puregene; Gentra Systems, Minneapolis, Minn.). Polymerase chain reaction was carried out as previously described. [Johnson, J. A. et al. (2003) “Beta 1-adrenergic receptor polymorphisms and antihypertensive response to metoprolol” Clin. Pharmacol. Ther. 74:44-52]. Genotypes were determined by pyrosequencing technology using a Pyrosequencing PSQ HS 96 system (Pyrosequencing AB, Uppsala, Sweden.). Haplotypes were inferred with double heterozygotes assigned as Ser49Gly389 and Gly49Arg389. This assignment was based on the absence of Gly49Gly389 alleles in over 1,200 alleles in whom β₁AR diplotype could be assigned in a previous study, despite being predicted in 52 alleles. Additionally, computational assessment of β₁AR haplotype revealed no Gly49Gly389 alleles in large population. [Terra, S. G. et al. (2002) “Beta1-adrenergic receptor polymorphisms and linkage disequilibrium (abstr)” Clin. Pharmacol. Ther. 71:P70].

The primary endpoint was a composite endpoint of “poor tolerability” defined as the need to discontinue metoprolol CR/XL or an inability to reach the target dose of 200 mg/day by the end of the titration period. Decompensated heart failure was a secondary composite endpoint defined as the composite of death or cardiac transplant, hospitalization for worsening heart failure, increase in other heart failure medications for worsening symptoms (e.g., diuretics), or need to discontinue metoprolol CR/XL for any reason during the titration period. The incidence of the decompensation endpoint was assessed during the titration period (i.e., 8-10 weeks) only. In addition to the composite endpoint, tolerability to metoprolol CR/XL was also assessed by scores on the MLWHF questionnaire, distance covered in the 6-minute walk test, and dose of metoprolol CR/XL attained by end of titration period. These endpoints were compared by genotype group for the polymorphisms in the β₁AR gene.

Patients were compared by genotype relative to the decompensation endpoints using a Chi square or Fisher's Exact test, as appropriate. Patients were also divided by genotype and compared for continuous parameters (e.g. MLWHF scores, 6-minute walk distance, dose of metoprolol CR/XL at end of titration, systolic blood pressure, heart rate) as well as changes in these parameters. Comparisons were made by unpaired t-test, ANOVA or Kruskal Wallis test, as appropriate. Discrete variables are listed as frequency counts and percentages while continuous variables are described as means ±SD or median (interquartile range). Polymorphisms that resulted in a homozygous variant genotype frequency of <2% were combined with the heterozygotes for analysis.

All statistical analyses were performed with the use of SAS (version 8; SAS institute INC, Cary, N.C.). A p-value of less than 0.05 was considered statistically significant.

Table 1 shows the clinical characteristics of the 61 patients enrolled in this study with data stratified by the β₁AR genotypes at codons 49 and 389 as these polymorphisms were the primary focus of the study. There was one subject in whom determination of the codon 49 genotype was unsuccessful. The Arg389 allele frequency was 0.72; there were 32 heterozygotes and one Gly389 homozygote. The Ser49 allele frequency was 0.85; there were 17 heterozygotes and no Gly49 homozygotes. None of the baseline characteristics listed in Table 1 were statistically significant between genotype groups. More Gly389 carriers were receiving digoxin and furosemide at baseline compared to Arg389 homozygotes, but the difference was not statistically significant (p=0.07 and 0.12, respectively). Patients were predominantly middle-aged Caucasian men, approximately 35% of patients had NYHA functional class III heart failure at enrollment and mean ejection fraction was 22±7%.

A similar percentage of patients in each β₁AR genotype failed to reach the target dose of metoprolol CR/XL or required discontinuation of therapy during the titration of therapy. Overall, 19/28 (67%) of Arg389 homozygous patients experienced “poor tolerability” to metoprolol CR/XL compared with 25/33 (75%) of Gly389 carriers (=0.62). For the codon 49 polymorphism, the frequency of “poor tolerability” was not different between genotype groups (72% and 76% for the Ser49Ser and Gly49 carriers, respectively).

Table 2 lists the endpoints for decompensated heart failure for the codon 49 and 389 genotype groups. There was a trend for Gly389 carriers to have a higher incidence of the composite endpoint of decompensated heart failure compared to Arg389 homozygotes within 8-10 weeks of initiating metoprolol CR/XL. This was largely the result of significantly greater percentage of Gly389 carriers requiring increases in other heart failure medications (mostly diuretics) for symptoms of worsening heart failure during β-blocker titration (48% vs. 14%, respectively; p=0.006). Although not statistically significant, patients with the Ser49 homozygous genotype had a nominally higher rate of all-cause decompensation compared with Gly49 carriers. This finding was driven largely by a significantly higher proportion requiring increases in heart failure medications to treat worsening symptoms (41% vs. 11%, respectively; p=0.03).

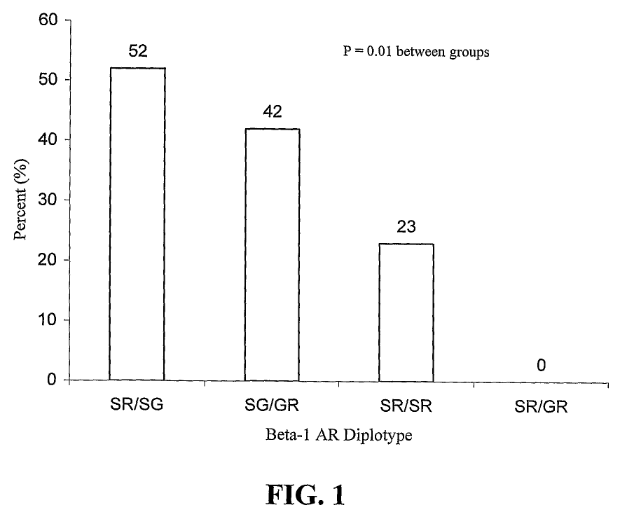

β₁AR diplotypes (based on codon 49 and 389 polymorphisms) were able to be determined in 60 patients. The observed diplotypes were SR/SG (Ser49Ser+Arg389Gly; n=25), SR/SR (Ser49Ser+Arg389Arg; n=17), SR/GR (Ser49Gly+Arg389Arg; n=10), SG/GR (Ser49Gly+Arg389Gly; n=7), and SG/SG (Ser49Ser+Gly389Gly; n=1). FIG. 1 shows there was a significant difference in the percentage of patients who required increases in heart failure medications when the response was stratified by the four most commonly observed β₁AR diplotypes (p=0.01). The presence of a Gly389 variant was associated with an increased likelihood of needing increases in concomitant heart failure therapy, while the presence of a Gly49 variant was associated with a decreased likelihood of needing increases in concomitant heart failure therapy. In all, 52% of patients with the SR/SG diplotype (Ser49Ser+Arg389Gly) and 42% of patients with the SG/GR diplotype (Ser49Gly+Arg389Gly) required increases in heart failure medications during metoprolol CR/XL titration. In contrast, only 23% of patients with the SR/SR diplotype (Ser49Ser+Arg389Arg) and 0% of patients with the with the SR/GR diplotype (Ser49Gly+Arg389Arg) required increases in heart failure medications during metoprolol CR/XL titration. Thus, the haplotype pair (diplotype) group with the most favorable tolerability of beta-blocker therapy was SR/GR. This is in contrast, and unpredicted from the hypertension studies (Johnson, et al.), where the most responsive diplotype was SR/SR.

Tolerability to metoprolol CR/XL was also assessed by measuring submaximal exercise tolerance (6-minute walk distance), scores on the MLWHF, and dose of metoprolol CR/XL achieved at the end of the titration period. Table 3 lists these data for the β₁-AR polymorphisms. At baseline, there were no significant differences in any of these parameters between the codon 49 and codon 389 genotypes. However, Gly49 carriers tended to cover a greater distance in the baseline 6-minute walk test compared to Ser49 homozygous patients. At the end of the titration period, there was a trend for Arg389 homozygous patients to have a greater exercise tolerance compared with Gly389 carriers. Scores on the MLWHF questionnaire and reduction in heart rate and systolic blood pressure were similar between β₁AR genotype groups (Table 3). There were no differences in metoprolol CR/XL dose at the end of the titration period between genotype groups (Table 3). In all, 67% of patients with the Arg389Arg genotype attained metoprolol CR/XL doses ≧100 mg/day compared to 51% of Gly 389 carriers (p=0.30). A similar percentage of patients reached a final dose of ≧100 mg/day (58% in each codon 49 group).

Based on these data, heart failure patients with the Gly389 variant and those with the Ser49 homozygous genotype are significantly more likely to require increases in heart failure medications during β-blocker titration than patients with other β₁AR genotypes. Furthermore, the combination of the codon 49 and 389 polymorphisms (i.e., β₁AR diplotypes) is also associated with a significantly higher risk for increases in concomitant heart failure medications indicating that these patients may require more frequent follow-up during beta-blocker titration.

### EXAMPLE 2

### ADRB1 (β₁AR Gene) and Left Ventricular Remodeling Changes in Response to Beta-Blocker Therapy

Patients were recruited for this prospective study from the University of Florida and University of North Carolina Heart Failure Clinics. All clinicians involved in clinical decision making were blinded to the genotypic data and all investigators determining the genotypes were blinded to the patient's clinical course. The study was approved by the Institutional Review Board at each institution, and each patient gave written informed consent for participation in the study. The procedures followed were in accordance with institutional guidelines. The study population included patients aged ≧18 years with symptoms of heart failure in NYHA functional class II-III, and with an EF≦40%. Patients had to have been receiving stable doses of an ACE inhibitor or angiotensin receptor blocker for ≧4 weeks. Patients were not eligible for enrollment if they were receiving β-blocker therapy. Other exclusion criteria were NYHA class IV, systolic blood pressure <90 mm Hg, heart rate <55 beats/minute, bronchospastic lung disease, >1^{st} degree heart block, active myocarditis, hypertrophic obstructive cardiomyopathy, and treatment with sotalol.

After obtaining informed consent, a 5-mL blood sample for isolation of genomic DNA was collected. Baseline evaluations included a two-dimensional (2D) quantitative echocardiogram for determination of measures such as ejection fraction (EF}, left ventricular end-diastolic diameter (LVEDD), LV end-systolic diameter (LVESD), and completion of a 6-minute walk test. After completion of baseline studies, patients began metoprolol CR/XL. The starting dose of metoprolol CR/XL was 12.5 mg/day for patients in NYHA class III and 25 mg/day for patients in NYHA class II. The dose was doubled on a biweekly basis to 200 mg/day or maximum tolerated dose. Thus, the time for the up-titration period was 8-10 weeks depending on the starting dose of metoprolol CR/XL. Patients underwent a follow-up echocardiogram three months after the attainment of completion of up-titration or attainment of the maximum metoprolol CR/XL dose. Thus, at the time of the follow-up echocardiogram, patients had been receiving metoprolol CR/XL for approximately 5-5.5 months. In a subset of 42 patients who consented to additional blood draws, a pharmacokinetic blood sample was obtained approximately 2 to 4 hours following dosing after two weeks on each metoprolol CR/XL dose for determination of S-metoprolol (active isomer) concentrations.

Echocardiograms were analyzed by a single experienced echo-cardiologist (KKH), blinded to the genotype results and results of previous echocardiograms. This individual had no knowledge of the clinical course of the patients. Left ventricular EF was estimated by visual inspection using parasternal long and short axis views as well as apical four chamber and two chamber views. Left ventricular dimensions at the mid-cavity or chordal level were measured from digitized end diastolic and end systolic parasternal long axis images of the left ventricle.

Genomic DNA was isolated from whole blood using a commercially available kit (Puregene; Gentra Systems, Minneapolis, Minn.). Polymerase chain reaction was carried out as previously described. [Johnson, J. A. et al. (2003) “Beta 1-adrenergic receptor polymorphisms and antihypertensive response to metoprolol” Clin. Pharmacol. Ther. 74:44-52). Genotypes were determined by pyrosequencing technology using a Pyrosequencing PSQ HS 96 system (Pyrosequencing AB, Uppsala, Sweden). Haplotypes were inferred with double heterozygotes assigned as Ser49Gly389 and Gly49Arg389. This assignment was based on the absence of Gly49Gly389 alleles in over 1,200 alleles in whom β₁AR diplotype could be assigned in a previous study, despite being predicted in 52 alleles. Additionally, computational assessment of β₁AR haplotype revealed no Gly49Gly389 alleles in large population. [Terra, S. G. et al. (2002) “Beta1-adrenergic receptor polymorphisms and linkage disequilibrium (abstr)” Clin. Pharmacol. Ther. 71:P70].

Patients were divided by genotype and compared for continuous parameters (e.g., LVEF, LVEDD) at baseline as well as changes in these parameters. Comparisons were made by unpaired t-test, ANOVA, Kruskal Wallis test, or Wilcoxon rank sum as appropriate. In addition to the unpaired comparisons between genotype groups, analyses within groups were performed with a paired t-test. As there are known racial differences in the allele frequencies at codon 389 in the β₁AR gene [Moore, J. D. et al. (1999) “Racial differences in the frequencies of cardiac β₁-adrenergic receptor polymorphisms: Analysis of c145A>G and c1165G>C” Hum. Mutat. 14:271], the data was assessed to determine whether a race interaction was present. Discrete variables are listed as frequency counts and percentages while continuous variables are described as means ±SD or median (interquartile range). Polymorphisms that resulted in a homozygous variant genotype frequency of <2% were combined with the heterozygotes for analysis.

Linear regression models were used to examine the joint effects of covariates on end of study EF and LVEDD. The following variables were analyzed for inclusion into the model: patient age, baseline heart rate, heart failure etiology, treatment with spironolactone, antiplatelet therapy, digoxin, furosemide, final metoprolol CR/XL dose, codon 389 genotype, and codon 49 genotype. In addition baseline EF and baseline LVEDD were added for their respective regression models. A backward elimination method was used to determine the best fitting regression model. A p-value of 0.10 was used for inclusion into the model.

All statistical analyses were performed with the use of SAS (version 8; SAS institute INC, Cary, N.C.). A p-value of less than 0.05 was considered statistically significant.

A total of 61 patients were enrolled in this study. Seven patients were prematurely withdrawn from the study due to either adverse events or withdrawal of consent. The baseline clinical characteristics of the 54 patients who had evaluable baseline and follow-up echocardiograms are shown in Table 4. The Arg389 allele frequency was 0.70; there were 23 Arg389 homozygotes, 30 heterozygotes, and 1 Gly389 homozygote. There were no significant baseline differences between the codon 389 genotype groups in baseline age, heart rate, systolic blood pressure, heart failure etiology, or duration of heart failure. At baseline, a significantly greater percentage of Gly389 carriers were receiving furosemide (p=0.01) and digoxin (p=0.02). After six months of treatment, Arg389 homozygotes were receiving a higher daily dose of metoprolol CR/XL compared to Gly389 carriers, although this difference was not statistically significant. (136±66 vs. 104±74, respectively; p=0.11). The median (interquartile range) S-metoprolol plasma concentrations were not different between Arg389 homozygotes and Gly 389 carriers at the final metoprolol CR/XL dose [31 ng/mL (14-58) vs. 19 ng/mL (11-36); p=0.43]. The final heart rate in Arg389 homozygotes and Gly389 carriers was 69 bpm in both groups, and the percent heart rate reduction from baseline was also not different between the codon 389 polymorphisms (Table 2), suggesting no differences in the degree of β-blockade.

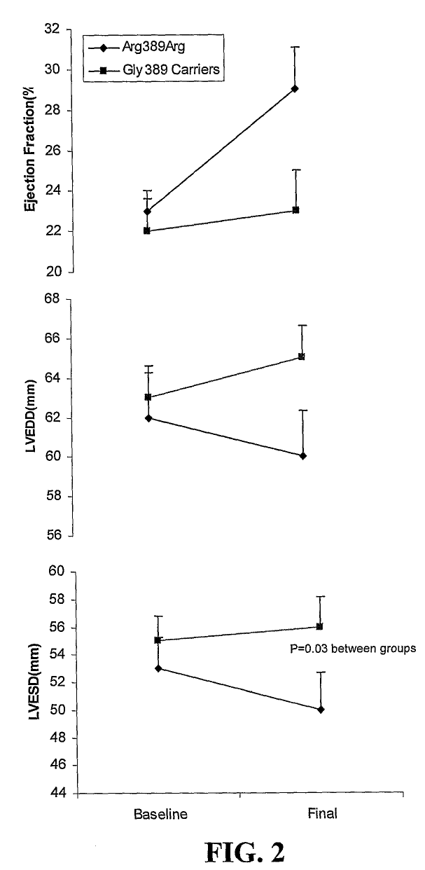

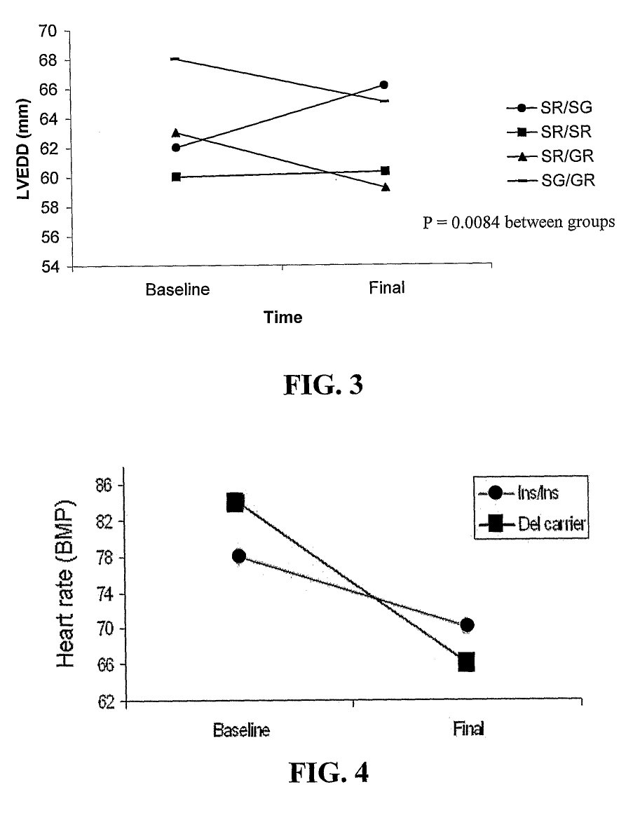

The echocardiogram results are summarized in Table 5 and FIGS. 2 and 3. For the codon 389 polymorphism, there were significant differences by genotype for the change in LVEF, LVEDD, and LVESD. For all three endpoints, patients with the Arg389 homozygous genotype had a greater improvement in LV remodeling. In contrast, patients who were Gly389 carriers did not have any significant change in their EF and in fact experienced a significant increase in the LVEDD during the study despite treatment with metoprolol CR/XL (Table 2 & FIG. 1). There was no significant interaction between race and codon 389 genotype with changes in LVEF, LVEDD, or LVESD (p-values=0.96, 0.82, 0.23, respectively). The codon 389 genotype was not associated with changes in heart rate or systolic blood pressure (Table 5).

The impact of the codon 49 genotype on changes in LVEF and remodeling was also evaluated. Codon 49 genotype was unable to be determined for one patient. The allele frequency for Ser49 was 0.86. There were 39 patients who were Ser49 homozygotes and 14 heterozygotes. At baseline, there were no statistically significant differences between Ser49 homozygous patients and Gly49 carriers (i.e., Ser49Gly) (data not shown). Following treatment with metoprolol CR/XL, Gly49 carriers experienced a significantly greater reduction in the LVEDD. In patients who carried the Gly49 variant, LVEDD decreased from 65±13 mm at baseline to 63±12 at six months (p=0.02). In contrast, for Ser49 homozygotes, LVEDD increased from 61±9 to 63±9 (p=0.01), with significant between-group differences (p=0.003). There were no other significant echocardiographic differences among patients when stratified by the codon 49 genotype. Left ventricular EF increased by an average of 2.8 units in both codon 49 genotype groups. The mean dose of metoprolol CR/XL was similar in both Ser49 homozygotes and Gly49 carriers (112±72 vs. 129±70, respectively; p=0.44).

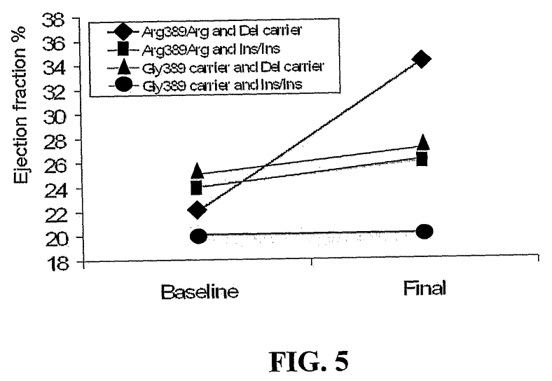

β₁AR diplotypes (based on codon 49 and 389 polymorphisms) were able to be determined for 53 patients. The observed diplotypes were SR/SG (Ser49Ser+Arg389Gly; n=24), SR/SR (Ser49Ser+Arg389Arg; n=14), SR/GR (Ser49Gly+Arg389Arg; n=8), SG/GR (Ser49Gly+Arg389Gly; n=6), and SG/SG (Ser49Ser+Gly389Gly; n=1). The one subject with the SG/SG diplotype was not included in the following analysis. The β₁AR diplotypes were significantly associated with changes in LVEDD (p=0.0084; FIG. 5). Patients with the SR/GR diplotype achieved a least squares mean decrease in LVEDD of 3.8 mm. Conversely, patients with the SR/SG diplotype experienced a least squares mean increase in LVEDD of 4.1 mm (FIG. 3). These data suggest that patients with SR/SG were actually harmed by beta-blocker therapy, since an increase in LVEDD would suggest a progression of the heart failure state. This was not predicted by the previous haplotype analysis in hypertensive patients, where the SR/SG genotype group was not the group with the worst response, and in fact had an intermediate blood pressure response to metoprolol. The difference between these two diplotypes was statistically significant (p=0.0032). β₁AR diplotypes were not significantly associated with changes in LVEF (p=0.17), LVESD (p=0.22), dose of metoprolol CR/XL (p=0.36), or differences in baseline heart rate (p=0.60). Furthermore, the change in heart rates from baseline were not significantly different by β₁AR diplotype (70 vs. 69 vs. 68 vs. 67 for SR/SG, SR/SR, SR/GR, and SG/GR, respectively (p=0.67).

Linear regression analysis was used to examine the combined effects of multiple variables on end of study EF and LVEDD. The variables that were significant predictors of a change in EF were baseline EF, use of spironolactone, use of furosemide, Arg389Arg genotype and metoprolol CR/XL dose (Table 6). Other variables including age, baseline heart rate and heart failure etiology were not significantly associated with changes in EF. Baseline LVEDD, heart failure etiology (ischemic etiology), Ser49Ser genotype, Arg389Arg genotype, and use of antiplatelet therapy were significant predictors of change in LVEDD (Table 7).

These data indicate that heart failure patients with the Arg389Arg genotype in the β₁-AR receptor gene experienced a greater increase in LVEF, and a larger improvement in LV remodeling as compared to Gly389 carriers. Secondly, patients with the Gly49 variant had a greater improvement in remodeling as measured by changes in LVEDD. In addition, β₁AR diplotypes that include the codon 49 and 389 polymorphisms demonstrated significant association with changes from baseline in LVEDD (FIG. 3). Further supporting these findings are the results of the multivariate analysis in which the codon 389 and codon 49 genotypes are significant predictors of change in LVEDD. Taken together, these results demonstrate that the codon 49 and 389 polymorphisms in the β₁AR gene are important determinants of LV remodeling changes in response to a β-blocker.

As predicted by the results of the individual genotypes, patients who carried both the Ser49Gly genotype along with the Arg389Arg genotype had the largest reduction in LVEDD. It is noted that the Ser49Ser genotype appears to have the potential to neutralize any beneficial effect of the Arg389Arg genotype. This finding is consistent with the multivariate analysis and the parameter estimates. As shown in Table 7, the Ser49Ser genotype predicted an increase in LVEDD of 6.23 mm. An earlier longitudinal study found that heart failure patients with the Gly49 variant appeared to have a lower mortality as compared to Ser49 homozygotes [Borjesson, M. et al. (2000) “A novel polymorphism in the gene coding for the beta(1)-adrenergic receptor associated with survival in patients with heart failure” Eur. Heart J. 21:1853-8]. The results of the diplotype analysis suggest that heart failure patients who are Gly389 carriers or those with the SR/SG diplotype (Ser49Ser+Arg389Gly) may not derive the same clinical benefit in response to a β-blocker.

These findings are also consistent with a previous report (14) showing that Arg389 homozygotes had a greater increase in EF than Gly389 homozygotes. However, the current data are more relevant to the heart failure population, as we show that Arg389 homozygotes also have a greater response than heterozygotes. The previous data suggested that approximately 6% of the population (Gly389Gly) might have a lesser improvement in EF. (14) These data extend those findings by showing that the same is true for heterozygotes, which represent an additional 35-40% of the population. Further, none of the previous studies in heart failure have consider the combination of the codon 49 and 389 genotypes (haplotypes), which is certain situation in this analysis were more informative than the single polymorphisms considered alone.

At baseline, a greater percentage of Gly389 carriers were receiving digoxin and furosemide. However, the genetic associations observed in this study are likely not due to these baseline differences for several reasons. First, traditional measures of disease severity such as EF, duration of heart failure, and distance covered in the 6-minute walk test were not significantly different at baseline. Therefore, there is no evidence that the Gly389 group was “sicker” at baseline relative to Arg389 homozygotes. Second, in the regression analysis, use of digoxin was not a significant predictor of change in EF or LVEDD. Third, use of furosemide predicted an increase in EF of 8.22 units. This suggests that the greater use of furosemide among Gly 389 carriers may have attenuated the actual differences in EF observed between the codon 389 genotype groups.

Although Arg389 homozygotes tended to receive a non-significantly higher dose of metoprolol CR/XL, it is not likely that this accounted for the differences in outcome measurements. First, the final heart rate in both codon 389 genotype groups was identical (69 bpm) indicating similar β-blockade between groups. Second, on multivariate analysis, the Arg389Arg genotype remained a significant independent predictor of an improvement in EF even after for controlling for metoprolol CR/XL dose. The regression model predicted that the Arg389 genotype was associated with an increase of 4.77 EF units. Finally, metoprolol CR/XL dose was not a significant predictor of change in LVEDD in the multivariate analysis, in contrast to the codon 389 and 49 polymorphisms, which significantly predicted changes in this outcome.

The above results allow one skilled in the art to select heart failure therapies based on individual genotypes.

### EXAMPLE 3

### Effects of β₁-α_{2C}-Adrenergic Receptor Polymorphisms on Ejection Fraction Response to β-Blocker Therapy in Heart Failure

Patients for the heart failure study were recruited for this prospective study from the University of Florida and University of North Carolina Heart Failure Clinics [Terra et al. (2005) “Beta1-adrenergic receptor polymorphisms and left ventricular remodeling changes in response to beta-blocker therapy” Pharmacogenet Genomics., 15:227-34]. In the hypertension study, patients were exclusively enrolled at the University of Florida [Johnson et al. (2003) “Beta 1-adrenergic receptor polymorphisms and antihypertensive response to metoprolol” Clin Pharmacol Ther., 74:44-52]. Populations and protocols for both prospective studies have been described in detail previously [Terra et al. (2005) “Beta1-adrenergic receptor polymorphisms and left ventricular remodeling changes in response to beta-blocker therapy. Pharmacogenet Genomics., 15:227-34; Johnson et al. (2003) “Beta 1-adrenergic receptor polymorphisms and antihypertensive response to metoprolol” Clin Pharmacol Ther., 74:44-52]. Both studies were approved by the Institutional Review Board at each institution, and each patient gave written informed consent for participation in each study. The study populations included mainly Caucasian and African American men and women. One study subject was of other ethnicity.

Heart failure Study. Inclusion criteria were age >18 years, NYHA functional class II-III, EF <40% and angiotensin-converting enzyme (ACE) inhibitor or angiotensin receptor blocker treatment on a stable dose for a minimum of four weeks. Patients receiving β-blocker therapy were not eligible. Other exclusion criteria included NYHA class IV, systolic blood pressure <90 mm Hg, treatment with sotalol, and have been described in more detail previously [Terra et al. (2005) “Beta1-adrenergic receptor polymorphisms and left ventricular remodeling changes in response to beta-blocker therapy” Pharmacogenet Genomics., 15:227-34].

At baseline, ejection fraction (EF), LV end-diastolic diameter (LVEDD) and LV endsystolic diameter (LVESD) were determined by a two-dimensional (2D) quantitative echocardiogram. Echocardiograms were analyzed by a single experienced echo-cardiologist. This individual had no knowledge of the clinical course or the genotyping results. Patients in NYHA class III started with metoprolol CR/XL 12.5 mg/day. The starting dose for patients in NYHA class II was 25 mg/day. The dose was doubled biweekly and time for the uptitration period was 8-10 weeks depending on the starting dose of metoprolol CR/XL. Echocardiography and 6 min walk test were repeated after patients had been maintained on the target dose (200 mg/day) or the highest tolerated dose for 3 months. Taken together the time patients received metoprolol CR/XL was approximately 5-5.5 month at the time the repeated echocardiography was performed.

Hypertension Study. In the hypertension study, 40 hypertensive men and women aged 35 to 65 years were studied. Hypertension was defined as untreated DBP between 95 mm Hg and 115 mm Hg (2 clinic visits, ≧5 minutes sitting). A certain range for systolic blood pressure (SBP) was not required for study entry. Patients being treated with antihypertensive medications had them tapered and then were assessed for hypertension after a 2 week drug-free period. Medications for other indications were only allowed if it could not conceivably affect BP. Other exclusion criteria included SBP >180 mm Hg, angina and congestive heart failure, and have been described previously [Johnson et al. (2003) “Beta 1-adrenergic receptor polymorphisms and antihypertensive response to metoprolol” Clin Pharmacol Ther., 74:44-52; Johnson et al. (1995) “Metoprolol minimizes nighttime blood pressure dip in hypertensive black males” Am J Hypertens., 8:254-9].

For baseline studies, blood samples were obtained for genotyping and 24-hour ambulatory blood pressure (ABP) monitoring was measured by using either an Accutracker DX device (SunTech Medical Instruments, Inc, Raleigh, N.C.) or an SpaceLabs 90207 oscillometric ABP monitor, (SpaceLabs Inc, Redmond, Wash.). Patients took 50 mg metoprolol every 12 hours and BP was measured every week at the same time of the day to minimize the impact of diurnal BP variation. To achieve the goal DBP <90 mm Hg, the dose could be increased weekly to 100 mg and the maximum of 200 mg every 12 hours. Doses were not increased if the patient had intolerable side effects. Patients who achieved goal DBP, as well as patients who did not reach DBP but had a reduction in DBP of ≧10%, continued therapy for a minimum of four weeks.

After the treatment phase, 24-hour ABP monitoring was repeated and blood samples for a pharmacokinetic analysis were taken. Detailed methods have been described previously [Johnson et al. (2003) “Beta 1-adrenergic receptor polymorphisms and antihypertensive response to metoprolol” Clin Pharmacol Ther., 74:44-52; Johnson et al. (1996) “Metoprolol metabolism via cytochrome P4502D6 in ethnic populations” Drug Metab Dispos., 24:350-5].

### Genotyping

Genomics DNA was isolated from whole blood using commercially available kits (Puregene; Gentra Systems Inc, Minneapolis, Minn. and Qiagen DNA Blood Isolation Kit, Qiagen, Valencia, Calif.) and normalized to 20 ng/mL. Genotyping methods for the Arg389Gly ADRB1 polymorphism have been described previously [Maqbool et al. (1999) “Common polymorphisms of beta1-adrenoceptor: identification and rapid screening assay” Lancet, 353:897; Terra et al. (2005) “Beta1-adrenergic receptor polymorphisms and left ventricular remodeling changes in response to beta-blocker therapy” Pharmacogenet Genomics., 15:227-34; Mason et al. (1999) “A gain-of-function polymorphism in a G-protein coupling domain of the human beta1-adrenergic receptor” J Biol Chem., 274:12670-4; Johnson et al. (2003) “Beta 1-adrenergic receptor polymorphisms and antihypertensive response to metoprolol” Clin Pharmacol Ther., 74:44-52]. The methods are based on polymerase chain reaction (PCR) followed by either pyrosequencing (heart failure study) or restriction fragment length polymorphism (hypertension study). Both methods are equally accurate [Aquilante et al. (2004) “Comparison of cytochrome P450 2C9 genotyping methods and implications for the clinical laboratory” Pharmacotherapy., 720-6]. Due to reduced genotyping costs, pyrosequencing was chosen to serve as the genotyping method in the later conducted heart failure study.

For ADRA2C Ins/Del genotyping a pyrosequencing method was developed. In brief, 5′-GTGGAGCCGGACGAGAGC-3′ as the forward primer and 5′-Biotin-GGCGCGACAGGAAGAACTC-3′ as the reverse primer were used for PCR amplification. The PCR mixture (10 μL) consisted of 0.1 μL Platinum™ Taq DNA Polymerase (Invitrogen, Carlsbad, Calif.), 0.25 μL PCR primers (10 μmol/μL), 0.8 dNTPs (25 mM), 2 μL 5× buffer F (Invitrogen), 1 μL of dimethyl sulfoxide, 3.6 μL of H₂O, and 40 ng of DNA. PCR was performed under the following conditions: 94° C. for 4 min; 42 cycles consisting of denaturation at 94° C. for 30 sec, annealing at 62° C. for 30 sec and extension at 72° C. for 30 sec; and final extension for 7 min. Genotypes were determined with pyrosequencing using the Pyrosequencing PSQ HS 96 System (Pyrosequencing AB, Uppsala, Sweden) and following the standard manufacturer protocol with (5′-GGTGCGGACGGGCAG-3′) as the sequencing primer and [GGGGCGGGGCCG]GGGGCGGCTGAGT as the sequence to analyze.

### Data Analysis

The analysis was based on the results of the two previous studies [Terra et al. (2005) “Beta1-adrenergic receptor polymorphisms and left ventricular remodeling changes in response to beta-blocker therapy” Pharmacogenet Genomics., 15:227-34; Johnson et al. (2003) “Beta 1-adrenergic receptor polymorphisms and antihypertensive response to metoprolol” Clin Pharmacol Ther., 74:44-52]. As an association with the Arg389Gly polymorphism with β-blocker response had been shown in both studies, the same association was tested for the ADRA2C Ins/Del polymorphism. Because the Del homozygous variant was expected to occur infrequently in the study population, it was planned a priori that these homozygotes would be combined with heterozygotes. Therefore, insertion homozygotes (Ins/Ins) were compared with Del carriers. In both the hypertension and heart failure study, baseline characteristics were compared between Ins/Ins and Del carriers by use of chi square test, Fishers exact test or Student's unpaired t-test, as appropriate. ADRA2C genotype was also tested for Hardy-Weinberg equilibrium by chi square test and transformation of variables, such as log AUC.

Hypertension Study. A priori power analysis revealed that 10 patients per genotype group (Ins/Ins or Del Carrier) were required to provide 80% power to detect a difference in DBP response of 6 mm Hg between genotypes with a 2-tailed a of 0.05.

The hypertension study showed that patients with Arg389Arg genotype had a better BP response. Daytime DBP reduction and 24-hour DBP reduction were significantly greater for Arg389Arg patients when compared to Gly389 carriers. Other BP responses showed a trend in favor for Arg389Arg but could not reach statistical significance [Johnson et al. (2003) “Beta 1-adrenergic receptor polymorphisms and antihypertensive response to metoprolol” Clin Pharmacol Ther., 74:44-52]. Thus, univariate linear regression was used to model the DBP responses (24-hour, daytime) with the Ins/Del variable. For further analysis, the Ins/Del variable could only enter a multivariate regression model if its P value would be less than 0.1 in the univariate model. This model would also include other, in the univariate model significant variables (P<0.1 for entry).

Heart failure Study. Based on α of 0.05 and 80% power, at least 20 patients per genotype group (Ins/Ins or Del Carrier) were required to detect a difference of 6 EF units. Our previous study showed that after treatment with metoprolol, EF increased significantly in patients with Arg389Arg genotype [Terra et al. (2005) “Beta1-adrenergic receptor polymorphisms and left ventricular remodeling changes in response to beta-blocker therapy” Pharmacogenet Genomics., 15:227-341. Gly389 carriers did not show a significant change. In addition, patients with Arg389Arg genotype experienced a significantly greater reduction in LVEDD and LVESD compared to Gly389 carriers. Therefore, univariate linear regression models were used to predict end of study EF, LVEDD, or LVESD with the Ins/Del variable. As in the analysis for the hypertension study, the Ins/Del variable entered the multivariate models if P was less than 0.1 in the univariate models. The multivariate linear regressions included also variables that were significant in the univariate models. Three different step-type selection methods (backward elimination, forward elimination, and minimum R² improvement) were used to determine the best-fitting regression model from the maximum model. Model entry was set at P<0.1 and variables with P<0.05 stayed for the next step.

If, in a final multivariate regression model, the Ins/Del polymorphism was significantly (P<0.05) associated with an outcome that had been associated with the Arg389Gly polymorphism (24 hour and daytime DBP; LVEF, LVEDD or LVESD), the study populations were divided in four groups: Arg389Arg and Del carrier, Arg389Arg and Ins/Ins, Gly389 carrier and Del carrier, and Gly389 carrier and Ins/Ins. Outcomes associated with both polymorphisms were then compared between groups using ANCOVA to adjust for other significant outcomes predictors. To test if the group with Arg389Arg and Del carriers improved markedly, t-tests were used comparing this group with the each of the three other groups. Here, dependent variables (24 hour and daytime DBP; LVEF, LVEDD or LVESD) were transformed (square root, logarithm) if they were not normally distributed. In addition, Benjamini-Hochberg correction was used for multiple comparison adjustment.

Statistical analyses were conducted using the Statistical Analysis Software (SAS) version 9.1 (Cary, N.C.) and P<0.05 was considered statistically significant. The level of significance was set to 0.1 only in the univariate models to avoid missing outcome predictors.

### Results

Sixty one patients were originally enrolled in the heart failure, but seven patients were withdrawn from the study due to either adverse events (e.g., inability to tolerate metoprolol) or withdrawal of consent. Therefore, 54 patients were available for analysis. All 40 patients completed the hypertension study and adverse effects from metoprolol in this population were minor. Baseline characteristics and genotype results for the two study populations are shown in Table 8. Genotyping data were completely available for ADRB1 and ADRA2C.

Heart failure Study. At baseline, Ins/Ins and Del carrier did not differ in age, gender, BMI, diabetes mellitus, New York Heart Association functional class, etiology, 6 min walk test, ADRB1 genotype and use of digoxin, spironolactone, and antiplatelet therapy. However, mean baseline heart rate was significantly higher in Del carrier when compared to Ins/Ins (84±9 BPM vs. 78±10 BPM; P=0.032). Del carriers were also younger (52 vs. 61 P=0.013) and more likely taking diuretics (100% vs. 79%, P=0.038). In addition, the Del allele was more prevalent among African Americans compared to Caucasians (0.50 vs. 0.09, P<0.0001). For both populations, the polymorphism was in Hardy-Weinberg equilibrium.

Due to beta-blockade, heart rate did not differ at study end comparing Del carriers with Ins/Ins (70±10 BPM vs. 67±11 BMP; P=0.245) and Del carriers improved significantly (−19±10 BPM) when compared with Ins/Ins (−7±11, P=0.002) (FIG. 4). The Ins/Del variable was not associated with change in LVEDD or LVESD. However, the Ins/Del variable was significantly associated with change in EF in a univariate regression model (P=0.025). Other significant variables in univariate models were final metoprolol CR/XL dose, use of spironolactone and Arg389Gly genotype. The multivariate predictors of the end study EF were baseline EF, Del-carrier status, Arg389Arg genotype and use of spironolactone (Table 9).

Sixteen heart failure patients were Arg389Arg and Ins/Ins homozygous, 13 were Gly389 and Del carrier, 7 had the combination Arg389Arg and Del carrier, and 18 were Gly389 carrier and Ins/Ins homozygous. The combination of Arg389Arg and Del-carrier showed the greatest EF change (FIG. 5). This group had an average 12 EF unit increase from a baseline EF of 22±6% to end study EF of 34±15%. An improvement by 2 EF units could be seen in the Arg389Arg/Ins/Ins or with Gly389/Del carrier groups. These groups' EF improved from 24±6% and 25±11% at baseline to 26±7% and to 27±15% at the end of the study, respectively. EF in the group with Gly389 carrier and Ins/Ins did not improve (baseline and end study EF: 20±6). Adjusting for the use of spironolactone, the change by 12 EF units in the Arg389Arg/Del carrier group was significantly different from all other combinations (Table 10).

Hypertension Study. Age, gender, BMI, smoking history, heart rate (baseline and study end), exercise (self-reported) and ADRB1 genotype were not different among Ins/Ins and Del carriers. Allelic frequencies differed among racial groups. Caucasians showed significantly lower frequency for the Del allele when compared to African Americans (0.10 vs. 0.50, P=0.008). The Chi-square statistic showed that both populations were in Hardy-Weinberg equilibrium.

In contrast to Arg389Gly, the Ins/Del variable was not a significant predictor for DBP responses (24-hour: P=0.34, daytime: P=0.38). Multivariate models would have also included smoking status and S-metoprolol plasma AUC (0-12). A trend toward a greater reduction in nighttime DBP or 24-hour, daytime or nighttime SBPs could not be detected between Ins/Ins or Del carriers. Nighttime DBP changed by −8.0±8.8 mm Hg in Ins/Ins (vs. Del carriers 3.7±9.7 mm Hg, P=0.19). In Ins/Ins, 24-hour SBP changed by −8.8±16.4 mm Hg (vs. Del carrier: −8.3±21.6, P=0.94), daytime SBP by −9.5±16.6 mm Hg (vs. Del carrier: −9.4±21.7, P=0.98), and nighttime SBP by −7.0±18.1 mm Hg (vs. Del carrier: −4.7±22.2, P=0.74).

### Discussion

In this study, the Ins/Del polymorphism in the ADRA2C gene and the Arg389Gly polymorphism in the ADRB1 gene were significant predictors of final EF in HF patients. The group with the combination Arg389Arg and Del carrier had a significantly greater improvement in EF compared to all other genotype combinations. Therefore, the Ins/Del polymorphism in the ADRA2C gene and the Arg389Gly polymorphism in the ADRB1 gene synergistically influence the left ventricular reverse remodeling response to β-blockers in HF.

Our findings are concordant with previous research. In transfected cells, the Arg389 polymorphism showed greater ability to couple to adenylyl cyclase compared to Gly389 and after agonist stimulation adenylyl activity levels were 3-4 higher [Mason et al. (1999) “A gain-of-function polymorphism in a G-protein coupling domain of the human beta1-adrenergic receptor” J Biol Chem., 274:12670-4]. In-vitro studies also associated the Del variant with reduced α_{2C}-adrenergic receptor function and increased norepinephrine release [Small et al. (2000) “A four amino acid deletion polymorphism in the third intracellular loop of the human alpha 2C-adrenergic receptor confers impaired coupling to multiple effectors” J Biol Chem., 275:23059-64; Small et al. (2001) “Identification and functional characterization of alpha(2)-adrenoceptor polymorphisms” Trends Pharmacol Sci., 22:471-7]. Therefore, the variants in both polymorphisms lead to higher activation of the sympathetic nervous system. Due to the complexity in the G-protein coupled receptors signal-transduction [Pierce et al. (2002) “Seven-transmembrane receptors” Nat Rev Mol Cell Biol., 3:639-50], this activation seems to be more than just a simple addition of each receptor effect. In addition, both receptors are present at two critical signaling pathways: The α_{2C}-adrenergic receptor acts as an autoreceptor of norepinephrine release control and the β₁-adrenergic receptor is the target receptor of norepinephrine.

Clinical studies have shown that ADRB1Arg389 homozygotes had a greater increase in EF than ADRB1 Gly389 homozygotes [Mialet Perez et al. (2003) “Beta 1-adrenergic receptor polymorphisms confer differential function and predisposition to heart failure” Nat Med., 9:1300-5] or Gly-carriers [Terra et al. (2005) “Beta1-adrenergic receptor polymorphisms and left ventricular remodeling changes in response to beta-blocker therapy” Pharmacogenet Genomics., 15:227-34]. In a recent study of patients with dilated cardiomyopathy, ADRA2C Del carriers had reduced events rates if treated according to guidelines [Regitz-Zagrosek et al. (2005) “α_{2C}-Adrenoceptor polymorphism is associated with improved event-free survival in patients with dilated cardiomyopathy” Eur Heart J., 27:454-9].

This study is the first to detect a synergistic effect of the Ins/Del polymorphisms and the Arg389Gly polymorphism in heart failure patients treated with β-blocker. In our heart failure study, the Ins/Del genotype was a significant predictor of end study EF and patients with both Arg389Arg and Del carrier status benefited substantially in more from β-blocker treatment in term of EF improvement. This improvement could not reach statistical significance (P=0.08) but a clear trend could be seen. Importantly, an EF increase of approximately 11% clearly represents a clinical improvement in LV function. In all other combination groups, EF increased by only 0-2%. As baseline EF was similar, patients with both Arg389Arg and Del carrier status experienced a 4-5 times greater EF improvement than patients in all other groups. Would the interaction between the Ins/Del polymorphisms and the Arg389Gly polymorphism just be an additive effect, one would expect that groups having either Arg389Arg or Del carrier would have an EF improvement of not 2% but 5-6%. This would be the expected mean between the two extreme observations of 11% EF improvement in the Arg389Arg and Del carrier group and 0% EF improvement in the Gly389 carrier and Ins/Ins group. The Ins/Del polymorphisms showed no association in the hypertension study. This lack of association highlights that the effects of various polymorphisms and their relationship with various disease states is unpredictable.

In addition, the response to β-blocker therapy is different in hypertension and heart failure. In hypertension, metoprolol lowers BP via multiple mechanisms, predominantly through suppression of renin release from the kidneys. How metoprolol exactly improves LV function in heart failure remains unclear, but inhibition of myocardial β₁-adrenergic overstimulation accounts for much of the benefit. As the adenylyl cyclase activity is enhanced in the Arg389 variant, a larger anti-adrenergic effect in patients with Arg389Arg could explain the improvements in LV function. However, metoprolol does not inhibit α-adrenergic receptors. An explanation might be that enhanced stimulation of cardiac α₁-adrenergic receptors in Del carriers leads to an increase in contractility and this improved EF. In addition, α_{1A/C}- and α_{1B} adrenergic receptors are required for physiological hypertrophy of normal cardiac development and for adaptive response in cardiac stress [O'Connell et al. (2003) “The alpha(1A/C)- and alpha(1B)-adrenergic receptors are required for physiological cardiac hypertrophy in the double-knockout mouse” J Clin Invest., 111:1783-91]. Therefore, heart failure patients with the Del variant could also have long-term benefits from β-blocker therapy. In particular, if these patients are also Arg389Arg homozygous.

TABLE 1
Baseline Demographic Characteristics Stratified by
β₁AR Genotype (Metoprolol Tolerability Study)

| | Arg389Arg | | Gly 389 Carriers | | Ser49Ser | | Gly 49 Carriers | |
|---|---|---|---|---|---|---|---|---|
| Variable | (n = 28) | | (n = 33) | | (n = 43) | | (n = 17) | |
| Age (years) | 59 ± 12 | | 55 ± 14 | | 58 ± 13 | | 55 ± 12 | |
| Men | 18 | (64%) | 19 | (57%) | 25 | (58%) | 11 | (64%) |
| Caucasian | 21 | (75%) | 21 | (63%) | 32 | (74%) | 10 | (58%) |
| NYHA FC, II/III | 17 (60%)/11 (40%) | | 22 (66%)/11 (34%) | | 25 (58%)/18 (42%) | | 13 (76%)/4 (23%) | |
| Ischemic HF | 7 | (25%) | 12 | (36%) | 15 | (34%) | 4 | (23%) |

| | | | | |
|---|---|---|---|---|
| BMI (kg/m²) | 27 ± 6 | 29 ± 6 | 27 ± 6 | 30 ± 6 |
| LV Ejection | 23 ± 5 | 22 ± 9 | 21 ± 7 | 25 ± 9 |
| Fraction (%) | | | | |
| Serum Sodium | 138 ± 3 | 138 ± 3 | 138 ± 4 | 139 ± 3 |
| (mEq/L) | | | | |

Concomitant Medical History

| | | | | | | | | |
|---|---|---|---|---|---|---|---|---|
| Diabetes | 7 | (25%) | 12 | (36%) | 14 | (32%) | 5 | (29%) |
| Hypertension | 14 | (50%) | 17 | (51%) | 21 | (48%) | 9 | (52%) |
| Atrial fibrillation | 6 | (21%) | 6 | (18%) | 11 | (25%) | 1 | (5%) |

Background Therapy

| | | | | | | | | |
|---|---|---|---|---|---|---|---|---|
| ACE | 28 | (100%) | 33 | (100%) | 43 | (100%) | 17 | (100%) |
| Inhibitor/ARB | | | | | | | | |
| Furosemide | 22 | (78%) | 31 | (93%) | 38 | (88%) | 14 | (82%) |
| Digoxin | 18 | (64%) | 28 | (84%) | 33 | (76%) | 13 | (76%) |
| Spironolactone | 9 | (32%) | 8 | (24%) | 12 | (28%) | 5 | (29%) |

Data are mean ± SD, or n (%)
There were no significant differences between genotype groups in any parameter

TABLE 2
Risk of Decompensated Heart Failure Stratified by
β₁AR Genotype (Metoprolol Tolerability Study)

| | | | Gly389 | | | | | Gly49 | | |
|---|---|---|---|---|---|---|---|---|---|---|
| | Arg389Arg | | Carriers | | | Ser49Ser | | Carriers | | |
| | (n = 28) | | (n = 33) | | P | (n = 43) | | (n = 17) | | P |
| Decompensated Heart | 8 | (28%) | 16 | (48%) | 0.11 | 19 | (44%) | 5 | (29%) | 0.29 |
| Failure (Composite) | | | | | | | | | | |
| Death | 0 | | 0 | | — | 0 | | 0 | | — |
| Hospitalization for | 1 | (3.5%) | 2 | (6%) | 0.99 | 3 | (6%) | 0 | | — |
| Worsening Heart Failure | | | | | | | | | | |
| Increase Heart Failure | 4 | (14%) | 16 | (48%) | 0.006 | 18 | (41%) | 2 | (11%) | 0.03 |
| Medications | | | | | | | | | | |
| Discontinuation of | 3 | (10%) | 3 | (9%) | 0.99 | 3 | (6%) | 3 | (17%) | 0.33 |
| Metoprolol CR/XL | | | | | | | | | | |

n (%)

TABLE 3
Impact of β₁-AR Genotypes on the 6-Minute Walk Distance, Quality-of-Life Scores, Hemodynamics,
and Dose of Metoprolol CR/XL at End of Titration (Metoprolol Tolerability Study)

| | Arg389Arg | | Gly389 carriers | | Ser49Ser | | Gly49 carriers | |
|---|---|---|---|---|---|---|---|---|
| | (n = 28) | | (n = 33) | | (n = 43) | | (n = 17) | |
| | | End of | | End of | | End of | | End of |
| | Baseline | Titration | Baseline | Titration | Baseline | Titration | Baseline | Titration |
| Metoprolol CR/XL | | 119 ± 72 | | 100 ± 74 | | 107 ± 71 | | 108 ± 79 |
| Dose | | | | | | | | |
| 6-min walk | 330 ± 108 | 357 ± 127 | 301 ± 75 | 293 ± 102* | 298 ± 89 | 306 ± 111 | 346 ± 88^{†} | 359 ± 131 |
| distance (m) | | | | | | | | |
| MLWHF | 28 ± 21 | 21 ± 22 | 36 ± 27 | 30 ± 20 | 31 ± 25 | 25 ± 20 | 37 ± 25 | 31 ± 25 |
| Heart Rate (bpm) | 80 ± 12 | 64 ± 12 | 79 ± 10 | 70 ± 12^{‡} | 80 ± 11 | 68 ± 12 | 79 ± 12 | 65 ± 12 |
| Systolic Blood | 120 ± 18 | 110 ± 20 | 119 ± 17 | 111 ± 18 | 118 ± 18 | 110 ± 18 | 121 ± 17 | 111 ± 21 |
| Pressure | | | | | | | | |
| (mm Hg) | | | | | | | | |

Mean +/− SD
*p = 0.06 vs. Arg389Arg at end of titration,
^{†}p = 0.07 vs. Ser49Ser at baseline
‡p = 0.09 vs. Arg389Arg at end of titration

TABLE 4
Baseline Characteristics of the Patients According to
Codon 389 Genotype (Ventricular Remodeling Study)

| | Arg389 homozygotes | | Gly389 carriers | |
|---|---|---|---|---|
| Variable | (n = 23) | | (n = 31) | |
| Age (years) | 59 ± 13 | | 56 ± 13 | |
| Men | 14 | (60%) | 18 | (58%) |
| Caucasian | 16 | (69%) | 21 | (67%) |
| NYHA FC, II/III | 15 (65%)/8 (35%) | | 22 (71%)/9 (29%) | |
| Duration of HF (years) | 4 | (1.5, 6.5) | 3 | (1.1, 8) |
| 6-minute walk distance (m) | 319 ± 115 | | 290 ± 77 | |
| Ischemic HF, n (%) | 6 | (26%) | 12 | (38%) |
| Diabetes mellitus | 6 | (26%) | 12 | (38%) |

Background Therapy

| | | | | |
|---|---|---|---|---|
| ACE Inhibitor/ARB | 23 | (100%) | 31 | (100%) |
| Furosemide | 17 | (73%) | 30 | (96%)* |
| Digoxin | 13 | (56%) | 26 | (83%)^{†} |
| Spironolactone | 8 | (34%) | 8 | (25%) |
| Antiplatelet | 10 | (43%) | 20 | (64%) |

*p = 0.01;
^{†}p = 0.02;
all other variables were not statistically significant

TABLE 5
Heart Rate, Blood Pressure and Left Ventricular Changes Stratified
by Codon 389 Genotype (Ventricular Remodeling Study)

| | Arg389Arg | | | Gly389 Carriers | | | |
|---|---|---|---|---|---|---|---|
| | (n = 23) | | | (n = 31) | | | |
| | Baseline | Final | P* | Baseline | Final | P* | P^{†} |
| LV Ejection | 23 ± 5 | 29 ± 10 | 0.008 | 22 ± 9 | 23 ± 11 | 0.45 | 0.04 |
| Fraction (%) | | | | | | | |
| LV end- | 62 ± 11 | 60 ± 11 | 0.38 | 63 ± 9 | 65 ± 9 | 0.04 | 0.03 |
| diastolic | | | | | | | |
| diameter (mm) | | | | | | | |
| LV end- | 53 ± 11 | 50 ± 13 | 0.16 | 55 ± 10 | 56 ± 12 | 0.11 | 0.03 |
| systolic | | | | | | | |
| diameter (mm) | | | | | | | |
| Heart Rate | 82 ± 10 | 69 ± 10 | <0.001 | 78 ± 10 | 69 ± 10 | <0.001 | 0.34 |
| (bpm) | | | | | | | |
| Systolic Blood | 121 ± 18 | 117 ± 20 | 0.27 | 120 ± 16 | 114 ± 18 | 0.07 | 0.77 |
| Pressure | | | | | | | |
| (mm Hg) | | | | | | | |

*p-value for within group comparisons (baseline vs. final)
^{†}p-value for between group differences for change from baseline value
Data are mean ± SD

TABLE 6
Multivariate Analysis of Factors Influencing the Change
in Ejection Fraction (Ventricular Remodeling Study)

| | Parameter | | |
|---|---|---|---|
| Variable | Estimate | Partial R² (%) | P-value |
| Baseline Ejection Fraction | 1.08 | 52% | <0.0001 |
| Use of Spironolactone | 6.19 | 6% | 0.004 |
| Use of Furosemide | 8.22 | 5% | 0.005 |
| Arg389Arg | 4.77 | 4% | 0.02 |
| Metoprolol CR/XL Dose | 0.03 | 4% | 0.02 |

Ejection Fraction expressed in ejection fraction units
R² = 0.71; Intercept for the Model = −13.79
Final Ejection Fraction = −13.79 + 1.08 (baseline ejection fraction) + 6.19 (if on spironolactone) + 8.22 (if on furosemide) + 4.77 (if Arg389Arg) + 0.03 (metoprolol CR/XL dose)

TABLE 7
Multivariate Analysis of Factors Influencing the Change in
LV End-Diastolic Diameter (Ventricular Remodeling Study)

| Variable | Parameter Estimate | Partial R² (%) | P-value |
|---|---|---|---|
| Baseline LV End- | 0.82 | 62% | 0.008 |
| Diastolic Diameter | | | |
| Ischemic Etiology | −6.67 | 9% | <0.0001 |
| Ser49Ser | 6.23 | 6% | 0.0005 |
| Arg389Arg | −4.05 | 4% | 0.007 |
| Use of Antiplatlet | 2.42 | 2% | 0.09 |
| therapy | | | |

LV End-Diastolic Diameter expressed in millimeters
R² = 0.83; Intercept = 10.75; − sign indicates a decrease in LV end-diastolic diameter
Final LV End-Diastolic Diameter = 10.75 + 0.82 (baseline LV end-diastolic diameter) − 6.67 (if ischemic etiology) + 6.23 (if Ser49Ser) − 4.05 (if Arg389Arg) + 2.42 (if on antiplatelet therapy).

TABLE 8
Baseline characteristics, genotyping results

| Variable | Hypertension Study | | Heart Failure Study | |
|---|---|---|---|---|
| Number of Patients | 40 | | 54 | |
| Age (years) | 48 ± 8 | | 58 ± 13 | |
| Men | 24 | (60%) | 32 | (59%) |
| Caucasian | 30 | (75%) | 37 | (68%) |

| Body mass index (kg/m²) | 31 ± 5 | | 28 ± 7 |
|---|---|---|---|
| Current or past smoker | 23 | (58%) | — |

| NYHA FC, II/III | — | 37 (69%)/17 (31%) | |
|---|---|---|---|
| Duration of HF (years) | — | 5 | (1.0, 7.0) |
| Ischemic HF | — | 18 | (33%) |

Background Therapy

| | | | |
|---|---|---|---|
| ACE inhibitor/ARB | — | 54 | (100%) |
| Furosemide | — | 47 | (87%) |
| Digoxin | — | 39 | (72%) |
| Spironolactone | — | 16 | (30%) |
| Antiplatelet | — | 30 | (56%) |

Genotyping results
ADRB1

| | | | | |
|---|---|---|---|---|
| Arg389Arg | 22 | (55%) | 23 | (43%) |
| Arg389Gly | 18 | (45%) | 30 | (56%) |

| | | | |
|---|---|---|---|
| Gly389Gly | 0 | 1 | (2%) |

ADRAC

| | | | | |
|---|---|---|---|---|
| Ins/Ins | 29 | (72%) | 34 | (63%) |
| Ins/Del | 7 | (18%) | 16 | (30%) |
| Del/Del | 4 | (10%) | 4 | (7%) |

NYHA FC, New York Heart Association Functional Class; HF, heart failure; ARB, angiotensin receptor blocker; ADRB1, β₁-adrenergic receptor gene; ADRA2C, α_{2C}-adrenergic receptor gene; Data are given as mean ± SD, or n (%)

TABLE 9
Multiple regression analysis of factors influencing
the treatment ejection fraction

| Variable | Parameter estimate | Partial R² (%) | P-value |
|---|---|---|---|
| Baseline ejection fraction | 1.06 | 54% | <.0001 |
| Del carrier | 5.28 | 5% | 0.0109 |
| Arg389Arg | 4.61 | 5% | 0.0234 |
| Use of spironolactone | 4.37 | 3% | 0.0440 |

R² = 0.67; Intercept for the model = −3.97. Final ejection fraction = −3.97 + 1.06 (baseline ejection fraction) + 5.28 (if Del carrier) + 4.61 (if Arg389Arg) + 4.37 (if on spironolactone).

TABLE 10
Changes in EF stratified by genotype combination of α_{2C}-adrenergic
receptor Ins/Del polymorphism and β₁-adrenergic receptor Arg389Gly
polymorphism

| | | Baseline | Final | EF | |
|---|---|---|---|---|---|
| | | EF | EF | change | |
| Genotype combination | n | (%) | (%) | (%) | P* |
| Arg389Arg and Del- | 7 | 22 ± 6 | 34 ± 15 | 12 | — |
| carrier | | | | | |
| Arg389Arg and Ins/ | 16 | 24 ± 6 | 26 ± 7 | 2 | 0.0437 |
| Ins | | | | | |
| G389 carrier and | 13 | 25 ± 11 | 27 ± 15 | 2 | 0.0437 |
| Del-carrier | | | | | |
| G389 carrier and | 18 | 20 ± 6 | 20 ± 6 | 0 | 0.0033 |
| Ins/Ins | | | | | |

*Multiple comparison adjusted (Benjamini-Hochberg) P-value for EF change differences between Arg389Arg/Del-carrier group vs. all other combinations (after square root transformation and adjustment for use of spironolactone)

1. A method of selecting at least one individual for treatment with β-blockers for the improvement of left ventricular ejection fraction response comprising:
a) genotyping the β₁ adrenergic receptor (β₁AR) and the α_{2C} adrenergic receptor (ADRA2C) gene of at least one individual;
b) determining whether the genotype of the β₁AR gene of said at least one individual is Arg389Arg and the genotype of the ADRA2C gene is DEL; and
c) selecting said at least one individual for treatment with β-blockers if said exhibits the ARG389ARG and DEL genotype.

2. A method of predicting that an individual would obtain clinical improvement in left ventricular function as a result of β-blocker therapy or identifying at least one individual that would obtain a clinical improvement in left ventricular function as a result of β-blocker therapy comprising:
a) genotyping the β₁ adrenergic receptor (β₁AR) gene and the α_{2C} adrenergic receptor (ADRA2C) gene of at least one individual;
b) determining whether the genotype of the β₁AR gene of said at least one individual is Arg389Arg and the genotype of the ADRA2C gene is DEL; and
c) identifying or predicting that an individual exhibiting the ARG389ARG and DEL genotype is expected obtain a clinical improvement in left ventricular function as a result of β-blocker therapy.

3. A method of treating at least one individual having impaired left ventricular function comprising:
a) genotyping the β₁ adrenergic receptor (β₁AR) gene and the α_{2C} adrenergic receptor (ADRA2C) gene of at least one individual;
b) determining whether the genotype of the β₁AR gene of said at least one individual is Arg389Arg and the genotype of the ADRA2C gene is DEL; and
c) administering a β-blocker medication to said individual if the genotype of the β₁AR gene of said at least one individual is Arg389Arg and the genotype of the ADRA2C gene is DEL.

4. A method of treating an individual, comprising providing, on the basis of β₁ adrenergic receptor (β₁AR) genotype, an appropriate degree of monitoring during initiation of beta-blockade, wherein individuals having a Gly49 carrier genotype or an Arg 389 homozygous genotype are monitored less closely and wherein patients having a Ser49 homozygous genotype or Gly389 carrier genotype are monitored more closely.

5. A method of predicting a need for increased monitoring or treatment in a heart failure patient comprising genotyping the β₁ adrenergic receptor (β₁AR) and identifying the codons at positions 49 and 389 of said patient, wherein the presence of a Ser49Ser homozygous genotype or Arg389Gly variant is associated with a requirement for increased monitoring of said heart failure patient during titration with a β-blocker medication or an increase in concomitant heart failure therapy as compared to a patient having a Ser49Gly genotype or an Arg389Arg genotype.

6. A method of predicting an individual's tolerability to beta-blocker treatment, comprising determining the genotype of said individual at codon 49 or codon 389 of the gene encoding the β₁ adrenergic receptor (β₁AR).

7. A method of predicting an individual's responsiveness to beta-blocker treatment, comprising determining the genotype of said individual at codon 49 or codon 389 of the gene encoding the β₁ adrenergic receptor (β₁AR).

8. The method of claim 6, wherein said individual is a heart failure patient.

9. The method of claim 6, wherein said tolerability relates to the need to modify prescribed levels or types of other therapeutics in conjunction with initiation of beta-blocker treatment.

10. The method of claim 6, wherein said determining comprises determining the genotype of the β₁ adrenergic receptor (β₁AR) of said individual at codon 49 and wherein the presence of the Gly49 carrier genotype is indicative of decreased risk for complications associated with the initiation of beta-blocker therapy.

11. The method of claim 6, wherein said determining comprises determining the genotype of the β₁ adrenergic receptor (β₁AR) of said individual at codon 49 and wherein the presence of the Ser49 homozygous genotype is indicative of increased risk for complications associated with the initiation of beta-blocker therapy.

12. The method of claim 6, wherein said determining comprises determining the genotype of the β₁ adrenergic receptor (β₁AR) of said individual at codon 389 and wherein the presence of the Gly389 carrier genotype is indicative of increased risk for complications associated with the initiation of beta-blocker therapy.

13. The method of claim 6, wherein said determining comprises determining the genotype of the β₁ adrenergic receptor (β₁AR) of said individual at codon 389 and wherein the presence of the Arg389 homozygous genotype is indicative of decreased risk for complications associated with the initiation of beta-blocker therapy.

14. The method of claim 7, wherein said individual is a heart failure patient.

15. The method of claim 7, wherein said responsiveness relates to reverse remodeling of the heart.

16. The method of claim 7, wherein said determining comprises determining the genotype of the β₁ adrenergic receptor (β₁AR) of said individual at codon 49 and wherein the presence of the Gly49 carrier genotype is indicative of increased responsiveness to beta-blocker therapy.

17. The method of claim 7, wherein said determining comprises determining the genotype of the β₁ adrenergic receptor (β₁AR) of said individual at codon 49 and wherein the presence of the Ser49 homozygous genotype is indicative of decreased responsiveness to beta-blocker therapy.

18. The method of claim 7, wherein said determining comprises determining the genotype of the β₁ adrenergic receptor (β₁AR) of said individual at codon 389 and wherein the presence of the Gly389 carrier genotype is indicative of decreased responsiveness to beta-blocker therapy.

19. The method of claim 7, wherein said determining comprises determining the genotype of the β₁ adrenergic receptor (β₁AR) of said individual at codon 389 and wherein the presence of the Arg389 homozygous genotype is indicative of increased responsiveness to beta-blocker therapy.

20. A method of treating an individual, comprising providing, on the basis of β₁ adrenergic receptor (β₁AR) genotype, an appropriate therapeutic agent, wherein beta blocker medications are prescribed preferentially or in greater proportion to an individual having a Gly49 carrier genotype or an Arg 389 homozygous genotype and wherein patients having a Ser49 homozygous genotype or Gly389 carrier genotype are preferentially or in greater proportion prescribed alternative non-beta blocker therapeutics.
